(19) United States
(12) Patent Application Publication
(10) Pub. No.: US 2020/0100880 A1
(43) Pub. Date: Apr. 2, 2020

(54) DENTAL TRAUMA KIT

(71) Applicant: Jeffrey A. Scharf, Exton, PA (US)

(72) Inventor: Jeffrey A. Scharf, Exton, PA (US)

(21) Appl. No.: 16/148,275

(22) Filed: Oct. 1, 2018

(51) Int. Cl. A61C 19/02; A61C 5/00; A61C 13/15; A61F 17/00; A61C 5/62

(52) U.S. Cl. CPC A61C 19/02; A61C 5/007; A61C 19/004; A61C 2202/01; A61C 5/62; A61C 2204/002; A61F 17/00

(57) ABSTRACT

A compact, portable, self-contained dental trauma kit for on-site treatment and repair of dental avulsion, luxation and, subluxation in remote, non-dental and other emergent settings consists of a dental splinting material (“the band-aid”), dental resins (“the glue”), and a dental curing light (“the glue hardener”). This is everything necessary for the user to stabilize the traumatically involved teeth according to proper dental protocol in remote locations, without the need for any additional materials or other accessories. The use of the kit allows the patient to be treated rapidly, in less than thirty minutes, which drastically increases the survival rate of re-implanted or repositioned tooth or teeth.

### BACKGROUND OF THE INVENTION

Accidents leading to dental trauma are common occurrences for both adults and children. Oral trauma can result from many different causes, including athletic accidents, motor vehicle accidents, and work-related incidents. When oral trauma leads to a tooth being “knocked-out” (avulsed) or “loosened” (luxated or subluxated), The International Association of Dental Traumatology and knowledgeable, reputable dental experts in general, strongly suggest immediate re-implantation at the place of the accident and support the use of short-terra flexible splints for stabilizing replanted teeth for up to four weeks. Splinting is the best practice in order to maintain the tooth, to improve function and for the comfort of the patient. Appropriate and timely treatment can greatly increase the chances that the tooth fully recovers from the injury. The American Association of Endodontists has reported, in their published document, “The Treatment of Traumatic Dental Injuries,” that the time out of the socket for an avulsed tooth is the most critical factor in its survival. If replanted within thirty minutes, the prognosis for saving the tooth is good.

However, portable dental trauma kits which can only, practically be used in remote, non-dental settings to address immediate dental avulsions and luxations are non-existent. Some dental trauma kits for non-dental professionals (e.g. EMT, trainers, military) are available. For instance, Travelers Supply offers an emergency dental kit for consumer use in emergency situations. The kit includes items to help deal with a toothache, loss of filling, cap or crown, tooth fracture and gum irritation. The Dental Box can be purchased by a licensed medical doctor or licensed dentist and includes items needed for the emergency care of acute dental emergencies by a medical professional. The dental kits offered by Dentrauma include storage mediums for teeth and recommends a visit to a dental office.

Hanks Balanced Salt Solution maintains the pH level of the tooth during transport in order to maintain the viability of the ligament cells that keep teeth in the mouth in place. Milk naturally keeps the tooth at a balanced pH. It has proven to be a suitable option for preserving an avulsed tooth during transport. However, temperature and availability in emergency situations is a factor, so studies show that milk can be used as a transport solution for avulsed teeth only when the teeth can be kept on ice. Save-a-Tooth is a product available to the public that claims to preserve the viability of an avulsed tooth until splinting can be completed. Non-dental professionals also use dual-cure materials for temporary splints until the patient visits a dental professional.

Dental professionals will normally treat trauma victims in their offices using dental resins and curing lights designed only to be used in chair-side, dental office settings. Current curing lights are not portable and require recharging. The curing light is stored on its base to ensure the battery remains charged, so it is available when the clinician needs it. The curing light battery will typically provide enough power for a full day of use. The cost for most curing lights currently available on the market ranges from $400-1500 USD. As a practical matter, current curing lights are not suitable for use in remote, in the field settings.

In fact, there are no curing lights which would reliably allow for curing of dental composites in a remote setting after a long period of storage. This also prevents the treatment of many types of procedures in settings other than a dental office. For instance, hospitals do not routinely carry or stock any form of a dental curing light. There are dental offices that are not even equipped with curing lights. In these cases, when oral surgeons are called in for an emergency in an emergency room setting, they are required to perform the same splinting procedure through the use of ligating wires. This may take two hours or more and can lead to additional trauma for the patient.

As a result, despite this extensive background in and history of treating dental trauma, a complete dental trauma kit is non-existent for the immediate treatment of avulsion, luxations, and like injuries in the field, in non-dental settings and in dental offices in which emergency procedures are not routinely performed.

### SUMMARY OF THE INVENTION

It is thus the object of the present invention to overcome the limitations of prior dental trauma treatment means by providing a compact, portable, self-contained dental trauma kit for the on-site, immediate treatment and repair of dental avulsion and luxation in remote, non-dental settings for non-dental medical professionals (hospital, athletic field, EMT, ambulance, athletic trainers, etc.). The kit can also be used in medical and dental facilities which do not have adequate dental equipment. The kit consists of a dental splinting material (“the band-aid”), dental resins (“the glue”), and a dental curing light (“the glue hardener”). This is everything necessary for the user to stabilize the traumatically involved teeth according to proper dental protocol in remote locations, without the need for any additional materials or other accessories. The use of the kit allows the patient to be treated rapidly, in less than thirty minutes, which drastically increases the survival rate of re-implanted or repositioned tooth or teeth.

While dental splinting material and dental resin are commonly used in dental settings, the curing light used in the dental trauma kit of the present invention must not only have a strong light output, but it also must be portable with an LED angle which provides ready access to the mouth and effective illumination to cure resin. The curing light will have a simple electronic circuit and can be battery operated or disposable, with an irreplaceable battery.

Using the three components in the kit, the dental splinting material, the dental resin, and the dental curing light, a medical professional is able to conveniently re-implant avulsed and/or stabilize mobile teeth by fabricating a dental splint in a matter of minutes. If a patient has teeth that are knocked out or become loose, the practitioner is able to replace the teeth in the socket and/or reposition the teeth in the proper position and then place the dental splint across the affected teeth, as well as across healthy teeth. The entire splint holds the teeth in place until they heal, at which point the entire splint is removed.

Although this is a common procedure in a dental office for treating a multitude of dental ailments, including dental trauma, at the current time the present invention allows the procedure to be performed efficiently and effectively in the field.

The novel features which are considered as characteristic of the invention are set forth in particular in the appended claims. The invention, itself, however, both as to its design, construction and use, together with additional features and advantages thereof, are best understood upon review of the following detailed description with reference to the accompanying drawings.

### BRIEF DESCRIPTION OF THE DRAWINGS

FIGS. 1-3 show one embodiment of the components and of the packaging of the components of the dental trauma kit of the present invention.

FIG. 4 shows a second embodiment of the components and of the packaging of the components of the dental trauma kit of the present invention.

FIG. 5 is an example of the results of use of the dental trauma kit of the present invention on traumatized teeth.

### DETAILED DESCRIPTION OF THE INVENTION

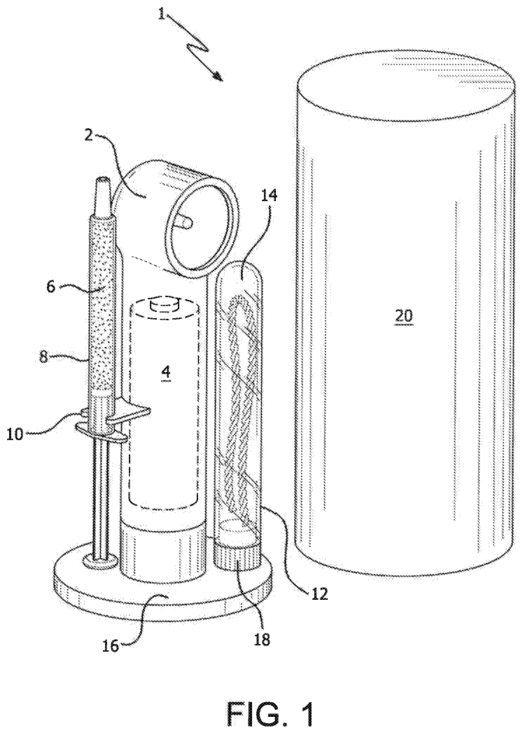

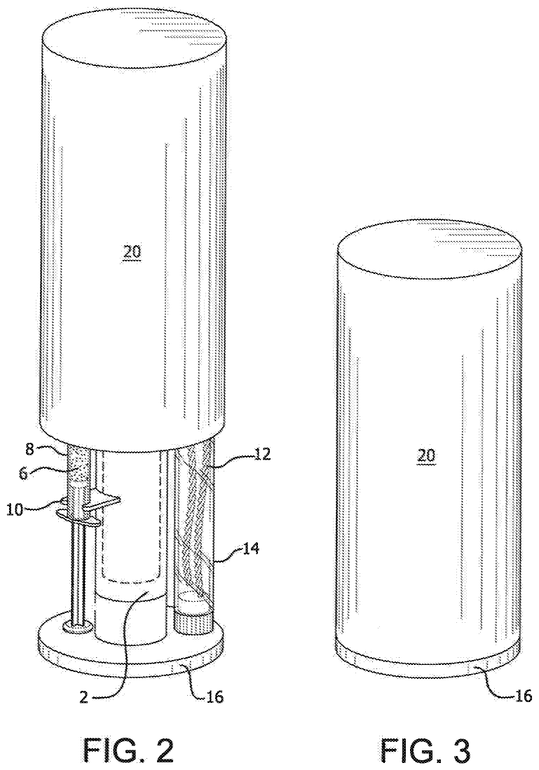

FIG. 1 shows the components of the present invention and how they may be packaged as a self-contained, compact, portable dental trauma kit 1. Kit 1 comprises curing light 2 containing electric battery 4, the electric circuitry of which is known in the art and is not a subject of this invention. Dental resin 6 in resin container 8 is held in place in relation to curing light 2 by bracket 10. Dental splinting material 12 is stored in splinting material holder 14, which, for example, may be secured to base member 16 via end cap 18, See FIG. 1. Dental splinting material 12 can be composed of glass, plastic, metal, or other material adapted to be used with resins. Cover 20 is configured to encircle curing light 2, resin container 8, splinting material holder 14, and base member 16. See FIGS. 2 and 3.

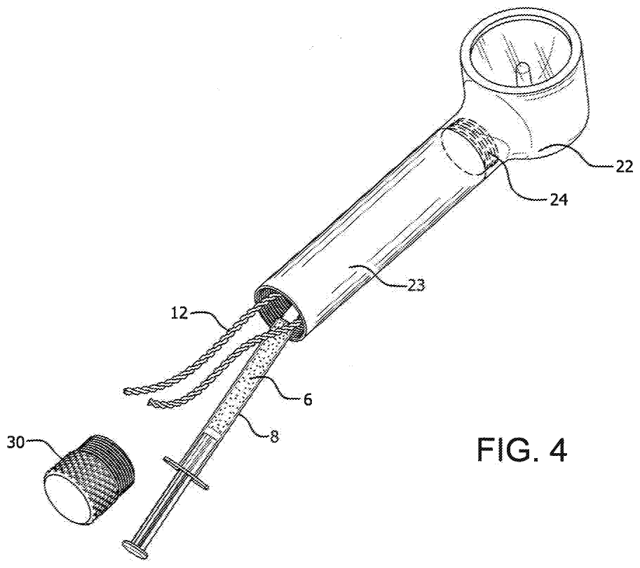

FIG. 4 shows a second embodiment of the dental trauma kit, in which curing light 22 comprises an elongated, main body 23 having an interior space in which is housed battery 24, dental resin 6 in resin container 8, and splinting material 12. Removeable end cap 30 is configured to be threadably attached to main body 23. As referenced previously, the electric circuitry of dental curing light 22 is known in the art and is not a subject of the invention. In this embodiment, dental curing light 22 with dental resin container 8 and dental splinting material 12 comprises a self-contained dental trauma kit.

The embodiments disclosed in FIGS. 1-3 and in FIG. 4 illustrate the components of the present invention, packaged together in a unique manner. However, the packaging of these components as a self-contained kit is not to be considered restricted to what is disclosed herein, as it is contemplated that other packaging configurations can be utilized.

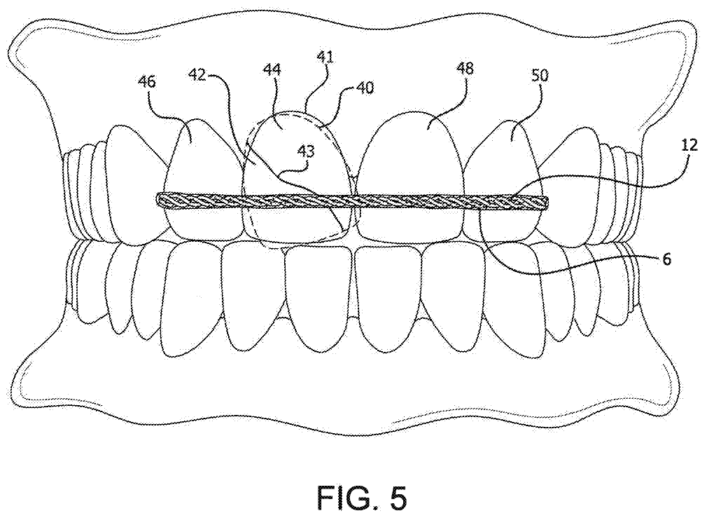

FIG. 5 illustrates an example of the results of properly utilizing the components of the dental trauma kit of the present invention. Following an accident, tooth segments 42 and 44 of cracked and luxated tooth 40 have immediately been positioned together. Crack 43 separates the tooth segments. Splinting material 12, with dental resin 6 having been applied, is laid over tooth 40, straighten and depicted as tooth 41, as well as over adjacent teeth 46, 48, and 50. Curing light 2 has been used to polymerize and harden resin 6 onto splinting material 12. This serves to stabilize tooth 41 and its adjacent teeth. Splinting material 12 will be allowed to remain over the four teeth until tooth segments 42 and 44 are permanently merged and the straighten position of tooth 41 is permanent, thus resulting in a solid tooth, properly set in place.

Certain novel features and components of this invention are disclosed in detail in order to make the invention clear in at least one form thereof. However, it is to be clearly understood that the invention as disclosed is not necessarily limited to the exact form and details as disclosed, since it is apparent that various modifications and changes may be made without departing from the spirit of the invention.

1. A compact, portable, self-contained dental trauma kit for immediate, on-site treatment and repair of dental avulsion, dental luxation, and dental subluxation, said kit consisting of:
a dental splinting material for placement directly on avulsed, luxated, or subluxated teeth for stabilizing said teeth;
a dental resin to be applied to the splinting material for attaching the splinting material to the teeth; and
a dental curing light for polymerizing the dental resin on the splinting material;
whereby the dental splinting material, the dental resin, and the dental curing light are specifically utilized on-site to immediately treat and stabilize the teeth without any additional materials, equipment, or accessories.

2. The dental trauma kit as in claim 1 further comprising a cover in which the dental splinting material, dental resin, and dental curing light are packaged.

3. The dental trauma kit as in claim 1 wherein the dental curing light comprises an elongated body enclosing an interior space, the body having a removable end cap.

4. The dental trauma kit as in claim 3 wherein the dental resin and dental splinting material are located in the interior space of the dental curing light body, such that the dental curing light with the dental resin and dental splinting material therein comprises the self-contained dental trauma kit.

5. The dental trauma kit as in claim 3 wherein the dental curing light comprises electric battery means located within the interior space for powering the dental curing light.

6. The dental trauma kit as in claim 4 wherein the dental curing light comprises electric battery means located within the interior space for powering the dental curing light.

7. The dental trauma kit as in claim 1 wherein said dental splinting material is composed of glass, plastic, metal, or other material adapted to be used with composite resins.
